(19) United States
(12) Patent Application Publication
(10) Pub. No.: US 2013/0225908 A1
(43) Pub. Date: Aug. 29, 2013

(54) Systems And Methods For A Magnetic Resonance Home Unit

(71) Applicants: Jerry I. Jacobson, Jupiter, FL (US); Allen Braswell, JR., Clearwater, FL (US); Frank Caruso, Golden, CO (US)

(72) Inventors: Jerry I. Jacobson, Jupiter, FL (US); Allen Braswell, JR., Clearwater, FL (US); Frank Caruso, Golden, CO (US)

(21) Appl. No.: 13/827,014

(22) Filed: Mar. 14, 2013

Related U.S. Application Data
(63) Continuation-in-part of application No. 12/546,385, filed on Aug. 24, 2009.
Application No. 12/546,385 is a continuation-in-part of application No. 12/500,284, filed on Jul. 9, 2009.
(60) Provisional application No. 61/091,582, filed on Aug. 25, 2008.
(60) Provisional application No. 61/079,670, filed on Jul. 10, 2008.

(51) Int. Cl. A61N 2/02

(52) U.S. Cl. CPC A61N 2/02
USPC 600/13; 600/9

(57) ABSTRACT

Methods, systems, computer-readable media, and apparatuses for a magnetic resonance home unit are presented. In one embodiment, a method for using a magnetic resonance home unit includes opening a magnetic resonance application; accessing a magnetic resonance treatment protocol associated with a magnetic resonance treatment; and outputting the magnetic resonance treatment to a patient.

### CROSS-REFERENCE TO RELATED APPLICATIONS

This application claims priority to and is a continuation-in-part of U.S. patent application Ser. No. 12/546,385, filed Aug. 24, 2009, and entitled “Systems and Methods for Providing a Magnetic Resonance Treatment to a Subject; (Attorney Docket PT102-57969/380065), which claims priority to Provisional Application No. 61/091,582, filed on Aug. 25, 2008, entitled “Systems and Methods for Providing a Magnetic Therapy Treatment to a Subject;” further, this application claims priority to and is a continuation in part of U.S. patent application Ser. No. 12/500,284, filed Jul. 9, 2009, entitled “Highly Precise And Low Level Signal-Generating Drivers, Systems, and Methods Of Use;” (Attorney Docket PT103-57969/378634), which claims priority to Provisional Patent Application No. 61/079,670, filed Jul. 10, 2008, and entitled “Highly Precise and Low Level Signal-Generating Drivers, Systems, and Methods of Use;” the entirety of each of which is incorporated herein by reference.

### TECHNICAL FIELD

The present disclosure generally relates to systems and methods for providing magnetic resonance to a subject. In particular, the present disclosure relates to a magnetic resonance therapy (MR) home unit and associated methods.

### BACKGROUND

Some patients may receive a magnetic resonance treatment. However, some magnetic resonance treatment devices are large and available only at provider's offices. Thus, a patient must travel away from his or her home. This may lead patients to skip treatments due to inconvenience. Thus, there is a need for a magnetic resonance home unit.

### BRIEF SUMMARY

Methods, systems, computer-readable media, and apparatuses for a magnetic resonance home unit are presented. In one embodiment, a method for using a magnetic resonance home unit includes opening a magnetic resonance application; accessing a magnetic resonance treatment protocol associated with a magnetic resonance treatment; and outputting the magnetic resonance treatment to a patient.

In a further embodiment, the magnetic resonance treatment is output in the patient's home.

In a further embodiment, the magnetic resonance application comprises a network connection to a remote database of magnetic resonance treatment protocols.

In a further embodiment, the magnetic resonance treatment protocol is stored in the remote database of magnetic resonance treatment protocols.

In a further embodiment, the magnetic resonance treatment protocol is developed by a magnetic resonance provider based on one or more prior magnetic resonance treatments to the patient.

In a further embodiment, the magnetic resonance treatment protocol is developed by a magnetic resonance provider based in part on a database data associated with other patients that have received magnetic resonance treatments.

In a further embodiment, the magnetic resonance treatment protocol is developed by the patient.

In a further embodiment, the magnetic resonance treatment comprises a magnetic field strength within the range of 1 gauss to 10⁻⁵⁰ gauss or other ranges within or outside of this range.

In one embodiment, a magnetic resonance home unit may comprise a magnetic resonance driver configured to receive a magnetic resonance treatment protocol, output a magnetic resonance treatment to a patient based in part on the magnetic resonance treatment protocol; a first coil assembly configured to be driven by the magnetic resonance driver, the first coil assembly comprising a base; a second coil assembly configured to be driven by the magnetic resonance driver, the second coil assembly comprising a base, the first and second coil assemblies configured to be collapsible.

In a further embodiment, magnetic resonance home unit is configured to be used by the patient in the patient's home.

In a further embodiment, the magnetic resonance driver is configured to receive the magnetic resonance treatment protocol from a magnetic resonance application.

In a further embodiment, the magnetic resonance application comprises a network connection to a remote database of magnetic resonance treatment protocols.

In a further embodiment, the magnetic resonance treatment protocol is stored in the remote database of magnetic resonance treatment protocols.

In a further embodiment, the magnetic resonance treatment protocol is developed by a magnetic resonance provider based on one or more prior magnetic resonance treatments to the patient.

In a further embodiment, the magnetic resonance treatment protocol is developed by a magnetic resonance provider based in part on a database data associated with other patients that have received magnetic resonance treatments.

In a further embodiment, the magnetic resonance treatment protocol is developed by the patient.

In a further embodiment, the magnetic resonance treatment comprises a magnetic field strength within the range of 1 gauss to 10⁻⁵⁰ gauss or other ranges within or outside of this range.

In one embodiment, a non-transient computer readable medium may comprise program code, which when executed by a processor is configured to cause the processor to: open a magnetic resonance application; access a magnetic resonance treatment protocol associated with a magnetic resonance treatment; and output the magnetic resonance treatment to a patient.

In a further embodiment, the program code is configured to execute a magnetic resonance application configured to access a network connection to a remote database of magnetic resonance treatment protocols.

These illustrative embodiments are mentioned not to limit or define the limits of the present subject matter, but to provide examples to aid understanding thereof. Illustrative embodiments are discussed in the Detailed Description, and further description is provided there. Advantages offered by various embodiments may be further understood by examining this specification and/or by practicing one or more embodiments of the claimed subject matter

### BRIEF DESCRIPTION OF THE DRAWINGS

Aspects of the disclosure are illustrated by way of example. In the accompanying figures, like reference numbers indicate similar elements, and:

FIG. 1 illustrates an isometric view of an MR home unit according to one embodiment of the present disclosure;

FIG. 2 illustrates an isometric view of the MR home unit of FIG. 1 with a user sitting therein according to one embodiment of the present disclosure;

FIGS. 3A and 3B, show illustrations of an expanded state and a collapsed state respectively of a magnetic resonance home unit according to one embodiment of the present disclosure;

FIG. 4 shows another embodiment of a magnetic resonance home unit according to one embodiment;

FIG. 5 shows another embodiment of a magnetic resonance home unit according to one embodiment;

FIG. 6 comprises an expanded view of one section of a magnetic resonance home unit according to one embodiment;

FIG. 7 shows another embodiment of a magnetic resonance home unit according to one embodiment;

FIG. 8 shows another embodiment of a magnetic resonance home unit according to one embodiment;

FIGS. 9A and 9B, show illustrations of an expanded state and a collapsed state of a magnetic resonance home unit according to one embodiment of the present disclosure;

FIG. 10 shows a block diagram of a magnetic resonance home unit according to one embodiment;

FIG. 11 shows another block diagram of a magnetic resonance home unit according to one embodiment;

FIG. 12 shows another block diagram of a magnetic resonance home unit according to one embodiment;

FIG. 13 shows another block diagram of a magnetic resonance home unit according to one embodiment;

FIG. 14 shows a flow diagram for using a magnetic resonance home unit according to one embodiment;

FIG. 15 shows another flow diagram for using a magnetic resonance home unit according to one embodiment; and

FIG. 16 shows another flow diagram for using a magnetic resonance home unit according to one embodiment.

### DETAILED DESCRIPTION

Several illustrative embodiments will now be described with respect to the accompanying drawings, which form a part hereof. While particular embodiments, in which one or more aspects of the disclosure may be implemented, are described below, other embodiments may be used and various modifications may be made without departing from the scope of the disclosure or the spirit of the appended claims.

### Illustrative Magnetic Resonance Home Unit

Magnetic resonance (MR) is a growing alternative treatment for many indications (e.g., illness, disease, medical condition, or ailment). A magnetic resonance device may be a natural and economical means of treating body pains and common injuries. In some embodiments, magnet resonance may be used to comfort or heal body areas, thereby avoiding the use of injections, pills, salves, or body-invasive procedures. A magnetic resonance treatment involves artificially produced magnetic fields. These fields interact with components such as, but not limited to, atomic or molecular components of living tissue, and then may have a beneficial effect on that living tissue.

Magnetic resonance may be provided by complementary and alternative medicine (CAM) healthcare providers (hereafter called service providers) that provide complementary and alternative medicine services to a patient population seeking active therapeutic treatment.

In some embodiments, the patient will travel to the practitioner's office to be treated. As a result, some clinical MR devices may be designed for office use. However, a challenge may exist in which when the MR treatment starts working (e.g., starts being effective) patients may choose to stop their treatment even though they could achieve greater benefit (e.g., greater relief of pain and/or other symptoms) by continuing treatment. For example, because some patients must physically travel to their practitioner's office and then pay for each treatment, the patient tends to abandon their MR treatment when a certain wellness level has been achieved even though it might not yet be the highest possible wellness level.

This inconvenience and cost can be overcome by providing MR treatment in one or more home treatment options. In some embodiments, these home treatment options may be provided using a system for a magnetic resonance home unit. In some embodiments, this home unit may be configured to provide a magnetic resonance treatment to the customer at his or her home. Further, in some embodiments, this magnetic resonance home unit may enable a customer to download data associated with a treatment regimen from a remote location. Further, in some embodiments, patients may be able to identify or create their own home treatment regimen.

Thus in some embodiments of the disclosure, a networked system for a magnetic resonance home unit includes at least one MR device that is operated by at least one practitioner in a clinical setting, a plurality of MR home units that are operated by the patients of the practitioner, and a centralized MR server that is accessible by both the practitioner for operating his/her MR device and the patients for operating their respective MR home units. In some embodiments, each of the MR home units is a home use MR device for whole-body immersion into precisely tuned, low-level electromagnetic fields for enhanced relaxation. In other embodiments, the MR home unit may comprise a home use MR device for immersion of body parts (e.g., arms, legs, hands, feet, knees, elbows, wrists, neck, head, etc.) into precisely tuned, low-level electromagnetic fields.

Additionally, in some embodiments, a MR network service provider may generate revenue for practitioners and provide service discounts to patients. For example, in some embodiments, patients may visit a practitioner to determine a treatment protocol. The patient may then receive the treatment using a magnetic resonance home unit. The patient may pay the service provider for the initial visit and to identify or create a treatment protocol. The patient may then subscribe to a magnetic resonance service provider that transmits data associated with the treatment protocol to the patient's magnetic resonance home unit, so that the patient may receive magnetic resonance treatment from home.

As used herein, the term “patient” means an animal, plant, object, and/or human, that may be the subject of the magnetic resonance treatment by use of the MR system of the present disclosure. A patient may include any user of the MR system in any location, for example at home. Similarly, the term “subject” is used herein to include a patient or any other person, animal, plant, or object upon which the MR system operates.

### Systems for a Magnetic Resonance Home Unit

A magnetic resonance therapy (MR) home unit and associated methods are disclosed. In some embodiments, the MR home unit may comprise a home use MR device for whole-body immersion into precisely tuned, low-level electromagnetic fields. In other embodiments, the MR home unit may comprise a home use MR device for immersion of body parts (e.g., arms, legs, hands, feet, knees, elbows, wrists, neck, head, etc.) into precisely tuned, low-level electromagnetic fields. Namely, the MR home unit disclosed herein may comprise a low cost, portable, lightweight, easy to ship, easy to assemble, and easy to use device. In some embodiments, patients can operate and treat themselves at home using the MR home unit, reducing or substantially eliminating the need to travel to or make appointments with a service provider.

Further, according to some embodiments, a Service Provider may determine a treatment protocol for a patient, or help a patient determine their own treatment protocol (either over the phone or internet, through trial and error, or based on a selection of one or more available treatment protocols.), the patient may use a magnetic resonance home unit to follow this treatment protocol. In some embodiments, the patient may subscribe to a treatment protocol. For example, the service provider could provide the patient with a subscription of varying treatment (e.g., field strengths, fluctuations, lengths of time, time of day, etc.) for a fixed monthly fee received from the patient.

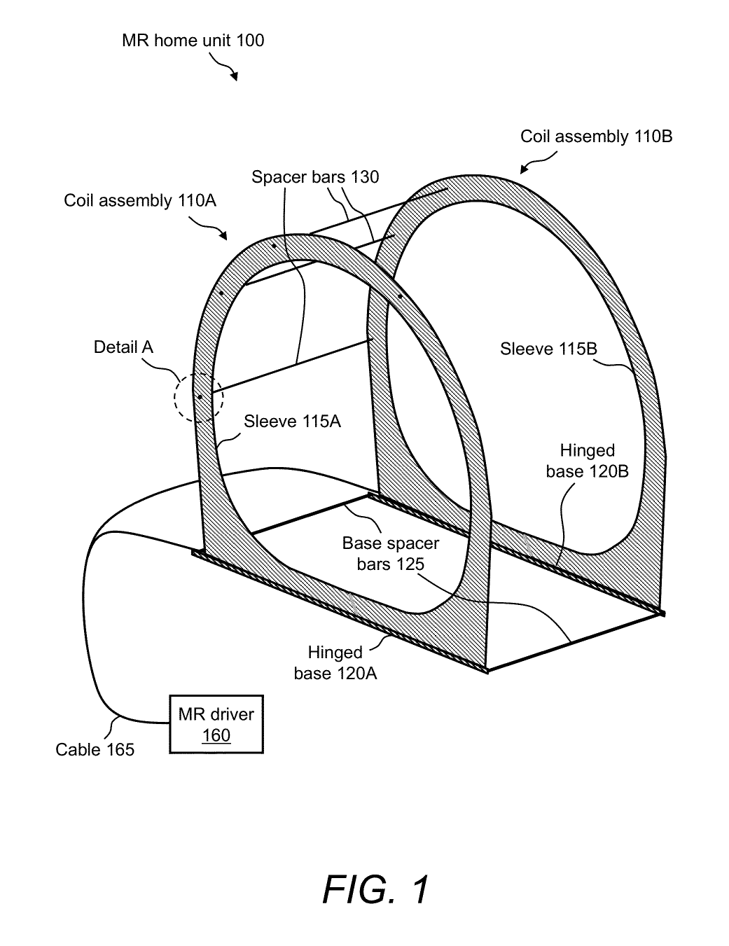

Turning now to FIG. 1, FIG. 1 illustrates an isometric view of an MR home unit according to one embodiment of the present disclosure. The system shown in FIG. 1 comprises an MR home unit 100, which comprises one example embodiment of an MR home unit device.

In the embodiment shown in FIG. 1, MR home unit 100 comprises a pair of coil assemblies 110 that may be driven by an MR driver 160. In the embodiment shown in FIG. 1, MR home unit 100 includes a coil assembly 110A and a coil assembly 110B. As shown in FIG. 1, each of the coil assemblies 110A, 110B is enclosed in a sleeve 115. For example, coil assembly 110A is enclosed in a sleeve 115A and coil assembly 110B is enclosed in a sleeve 115B. In the embodiment shown in FIG. 1, the coil assemblies 110, which include the sleeves 115, is mounted to a base 120. In some embodiments, base 120 comprises a hinged base. In the embodiment shown in FIG. 1, coil assembly 110A is mounted on hinged base 120A and coil assembly 110B is mounted on hinged base 120B. In other embodiments, base 120 may comprise a flexible or collapsible base. In still other embodiments, base 120 may not be an included component, and the coil assemblies may be coupled using for example, one or more spacer bars 130.

In the embodiment shown in FIG. 1, hinged base 120A, 120B are coupled together via a pair of base spacer bars 125. Additionally, in the embodiment shown in FIG. 1, a plurality of spacer bars 130 are arranged between coil assemblies 110A, 110B. By use of base spacer bars 125 and spacer bars 130, coil assemblies 110A, 110B are held upright and substantially parallel to one another. In one example embodiment, the coil assemblies 110A, 110B may be about 6 feet 6 inches high and be spaced about 3.5 feet apart. Further, in this example embodiment, base spacer bars 125 and spacer bars 130 are about 3.5 feet long. In some embodiments, base spacer bars 125 and spacer bars 130 can be, for example, hollow or solid aluminum rods or hollow or solid fiberglass rods. In some embodiments, the diameter of base spacer bars 125 and spacer bars 130 is from about 0.2 inches to about 0.5 inches. In another embodiment, the diameter of base spacer bars 125 and spacer bars 130 is about 0.25 inches. In another embodiment (not shown in FIG. 1), coil assemblies A and B may further, or alternatively supported by other braces, e.g., diamond, triangle, or X shaped supports that may be hinged, and may further allow for the folding of the device as described herein.

In the embodiment shown in FIG. 1, coil assemblies 110A, 110B of MR home unit 100 form a modified Helmholtz coil. In some embodiments, a Helmholtz coil may generate a substantially uniform homogeneous magnetic field between the two coils. Accordingly, when MR home unit 100 is operating, there is a substantially uniform homogeneous magnetic field between coil assemblies 110A, 110B. In some embodiments the magnetic field strength output as a part of the magnetic resonance treatment may be within the range of 1 gauss to 10⁻⁵⁰ gauss. In some embodiments, the magnetic resonance treatment may be in a sub-range within this range. In other embodiments, the magnetic resonance treatment may be at greater field strength than this range, e.g, 1, 2, or 3 gauss, or more.

As shown in FIG. 1, the coils in coil assemblies 110A, 110B are driven MR driver 160. A cable 165 may provide the electrical connection between MR driver 160 and coil assemblies 110A, 110B. In some embodiments, MR driver 160 and coil assemblies 110A, 110B may be remote from each other, and may comprise another type of connection, e.g., a wireless connection Further, in some embodiments, driver 160 may transmit signals to a sub-driver associated with coil assemblies 110A, 110B, which output magnetic resonance treatment based on these signals. MR driver 160 may be used to control the specific magnetic waveform parameters, such as waveform type (e.g., sinusoidal, rectilinear, square), amplitude (e.g., 1 mV), and frequency (e.g., 10 Hz) of the uniform homogeneous magnetic field that is generated between coil assemblies 110A, 110B.

In some embodiments, MR driver 160 is also used to control the duration of the MR treatment. In some embodiments, the settings of MR driver 160 are determined by a predetermined user-specific MR treatment protocol. Further, in some embodiments, the settings of MR driver 160 may be received from a remote source, for example, a database associated with a service provider, or a provider of subscriptions to MR treatments.

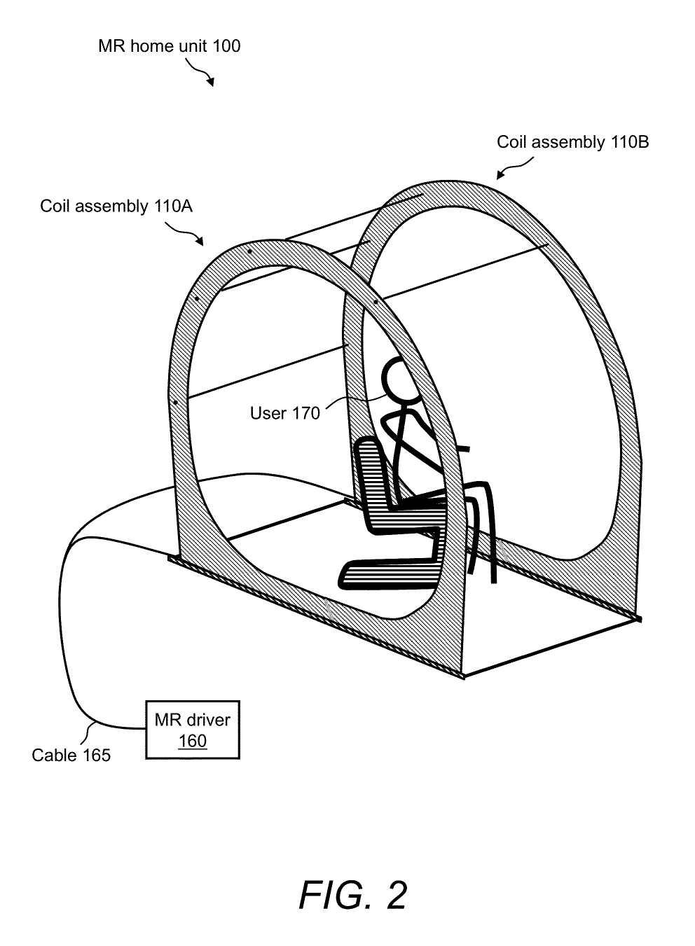

Turning now to FIG. 2, FIG. 2 shows MR home unit 100 described with regard to FIG. 1, with a user 170 sitting therein. In the embodiment shown in FIG. 2, user 170 is the subject of an MR treatment that is based on a MR treatment protocol. In some embodiments, the user may be in a different position. Similarly, in some embodiments, the MR treatment may be applied to a different subject, e.g., a non-human subject, such as an animal (dog, cat, lizard, bird, fish) a plant (e.g., a houseplant or crop), or some inanimate object (e.g., water or building materials).

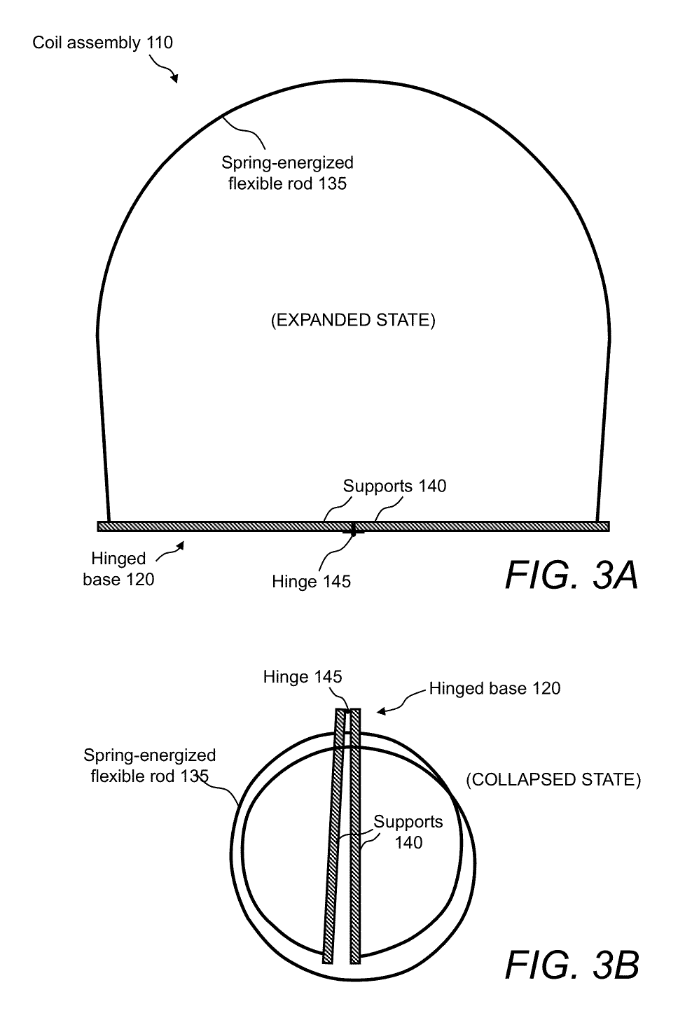

Turning now to FIGS. 3A and 3B, FIGS. 3A and 3B show illustrations of an expanded state and a collapsed state of one embodiment of a magnetic resonance home unit. FIG. 3A shows spring-energized flexible rod 135 in a fully expanded state and with hinge 145 locked. By contrast, FIG. 3B shows spring-energized flexible rod 135 in a collapsed state. For example, FIG. 3B shows that hinge 145 can be unlocked, which allows spring-energized flexible rod 135 to be folded into the small form factor shown in FIG. 3B (e.g., folded into a comparatively flat and smaller configuredation).

As shown in FIGS. 3A and 3B, in some embodiments, the purpose of hinged base 120 is to allow spring-energized flexible rod 135 and the other components of coil assembly 110 to be easily collapsed for easy packaging and shipment of MR home unit 100. Thus, as shown in FIG. 3A, hinge 145 is shown locked. In FIG. 3B, hinge 145 is shown unlocked, which allows the two supports 140 to fold together. At the same time that the two supports 140 are folded together, spring-energized flexible rod 135 can be twisted into a figure-eight, then the two loops of the figure-eight can be folding over one another to create the small form factor that is shown in FIG. 3B. For purpose of illustration, FIGS. 3A and 3B show only the spring-energized flexible rod 135 portion of the coil assembly 110. However, in other embodiments, not shown in FIGS. 3A and 3B, each fully assembled coil assembly 110 may be collapsible

In some embodiments, a magnetic resonance home unit may comprise a flexible spring-energized rod. As shown in FIGS. 3A and 3B, a first portion of a coil assembly 110 that includes a spring-energized flexible rod 135, the end of which are affixed to hinged base 120. In some embodiments, this spring energized flexible rod may comprise a flexible rod, configured to output a resilient force to oppose bending. In one embodiment, a spring-energized flexible rod 135 may comprise flexible fiberglass, steel, aluminum, wood, carbon, titanium, composite, or some other flexible material. In some embodiments, the diameter of spring-energized flexible rod 135 is from about 0.2 inches to about 0.5 inches. In other embodiments, the diameter of spring-energized flexible rod 135 is about 0.25.

Further, as shown in FIGS. 3A and 3B, hinged base 120 may be formed by two supports 140 that are coupled end-to-end via a hinge 145. In the embodiment shown in FIGS. 3A and 3B, hinged base 120 is therefore foldable. In some embodiments, supports 140 can be, for example, solid or hollow aluminum bars, solid or hollow rigid plastic bars, wooden bars, or laminated wood bars that are about 1 in cross-section.

Further, in some embodiments, hinge 145 may be lockable in the fully open position via any standard locking mechanism (not shown). In one embodiment, the length of spring-energized flexible rod 135 is about 16 ft in order to provide a coil assembly 110 that is about 6 feet 6 inches in height, suitable for whole-body immersion. In other embodiments, other sizes may be used for other types of subjects (e.g., plants, water, or objects). Further, other sizes may be configured to apply magnetic fields to body parts, e.g., hands, feet, arms, wrists, elbows, shoulders, legs, ankles, knees, neck, head, etc. For example, FIG. 3A shows spring-energized flexible rod 135 and hinged base 120 in a fully expanded state. In this state, hinge 145 is locked in the fully open position and spring-energized flexible rod 135 forms an arch that is about 6 feet 6 inches in height and about 7 feet in width. Further, in this example embodiment, each of the two supports 140 is about 3.5 feet long. In other embodiments, the components shown in FIGS. 3A and 3B may be of different thicknesses and lengths as needed for various size magnetic resonance home units.

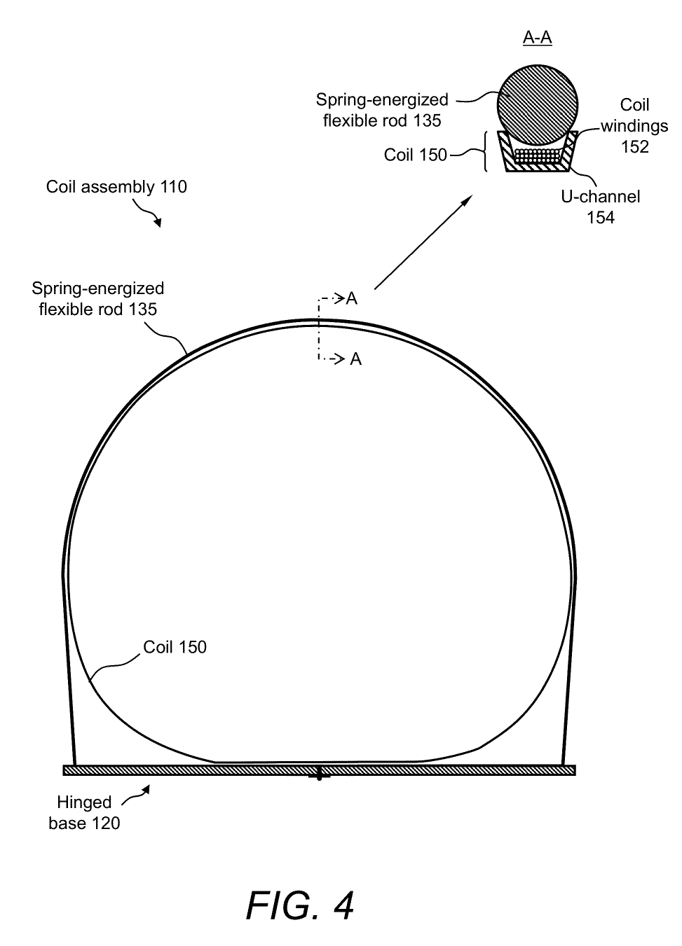

FIG. 4 shows another embodiment of a magnetic resonance home unit according to one embodiment. As shown in FIG. 4, each coil assembly 110 may further include a coil 150 that has a certain number of turns of a certain size of wire. For example, coil 150 may include 30 turns of 30-gauge solid-core copper wire. In another example, coil 150 includes 30 turns of 28-gauge solid-core copper wire. In some embodiments, this number of turns and the gauge and type of wire may be varied as needed based on the desired field strength and size of the magnetic resonance home unit. In one embodiment, if coil assembly 110 is about 6 feet 6 inches in height, then coil 150 may have a diameter of about 7 feet. However, the height of coil assembly 110 (and accordingly the diameter of coil 150) can vary depending on the overall size of magnetic resonance home unit 100.

FIG. 4 further shows a cross-sectional view taken along line A-A showing coil 150 in relation to spring-energized flexible rod 135. In the embodiment shown in FIG. 4, cross-section A-A shows coil windings 152 arranged in a U-channel 154. In this example, U-channel 154 is positioned against spring-energized flexible rod 135, wherein U-channel 154 may not be deep enough for spring-energized flexible rod 135 to be fitted therein. In this example, coil 150 can be affixed to spring-energized flexible rod 135 by, for example, a plurality of tie wraps. However, in another embodiment, for example, U-channel 154 can be made deep enough to accommodate coil windings 152 and also to snap-fit onto spring-energized flexible rod 135.

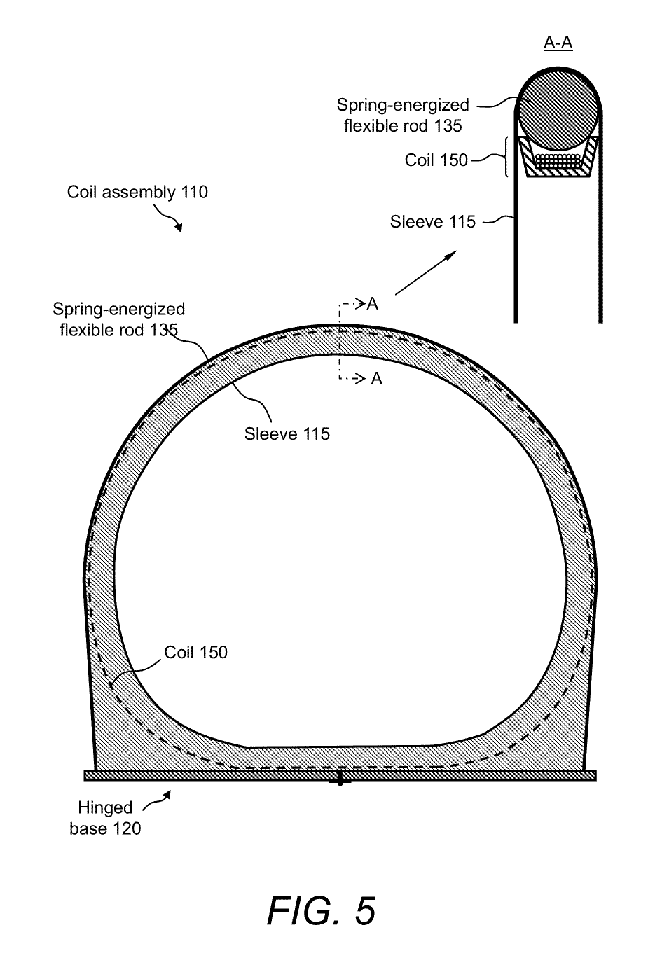

Referring now to FIG. 5, FIG. 5 shows another embodiment of a magnetic resonance home unit according to one embodiment. In the embodiment shown in FIG. 5, spring-energized flexible rod 135 and coil 150 are covered with a sleeve 115. A cross-sectional view taken along line A-A of FIG. 5 shows more details of coil 150 and spring-energized flexible rod 135 covered with sleeve 115. Sleeve 115 may be, for example, vinyl or ripstop nylon that is stretched over spring-energized flexible rod 135 and coil 150, as shown. In some embodiments, sleeve 115 is provided for both aesthetic and functional stability purposes and can therefore be any color, and made of many different types of materials. In the embodiment shown in FIG. 5, the coil assembly 110, which includes the combination of spring-energized flexible rod 135, coil 150, and sleeve 115, is collapsible or foldable as described above with reference to FIGS. 3A and 3B.

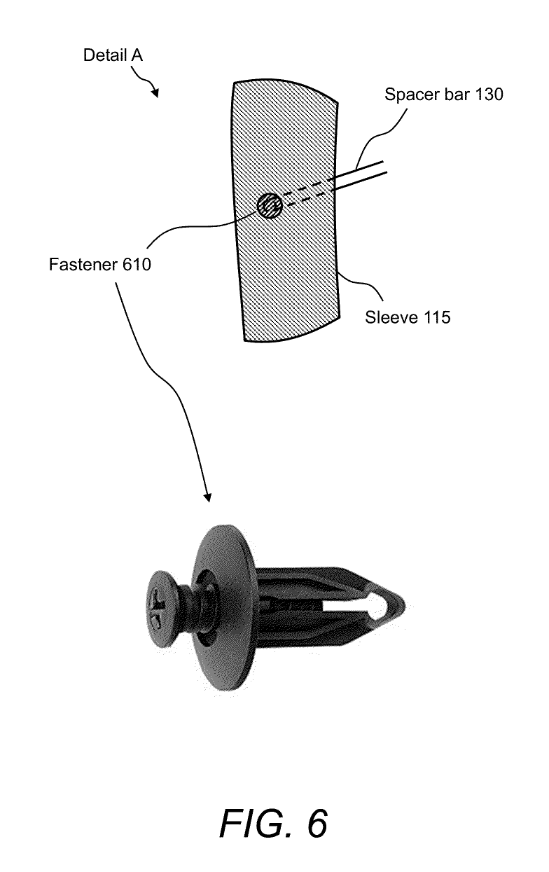

FIG. 6 shows an expanded view of one section of the system described with regard to FIG. 1. Specifically, FIG. 6 comprises one embodiment of an expanded view of a Detail A of FIG. 1. The embodiment shown in FIG. 6 comprises one example of a fastener that can be used in the MR home unit 100. For example, the ends of each of the spacer bars 130 may be affixed to sleeve 115 using a fastener similar to fastener 610.

In one embodiment, the spacer bar 130 is a hollow aluminum rod. In such an embodiment, a hole may be provided through the fabric of sleeve 115. Further, in such an embodiment, a fastener 610 may be pushed through the hole and into the hollow spacer bar 130. In this example, fastener 610 may comprise a plastic anchor that is inserted into the end of the hollow spacer bar 130 and then expanded by tightening a screw to provide a tight fit inside of the hollow spacer bar 130. In such an embodiment, the spacer bars 130 may be secured to sleeve 115. In other embodiments, MR home unit 100 is not limited to the type of fastener 610 shown in FIG. 6. For example, in some embodiments any type of fastener capable of securing the ends of spacer bars 130 to sleeves 115A, 115B can be used.

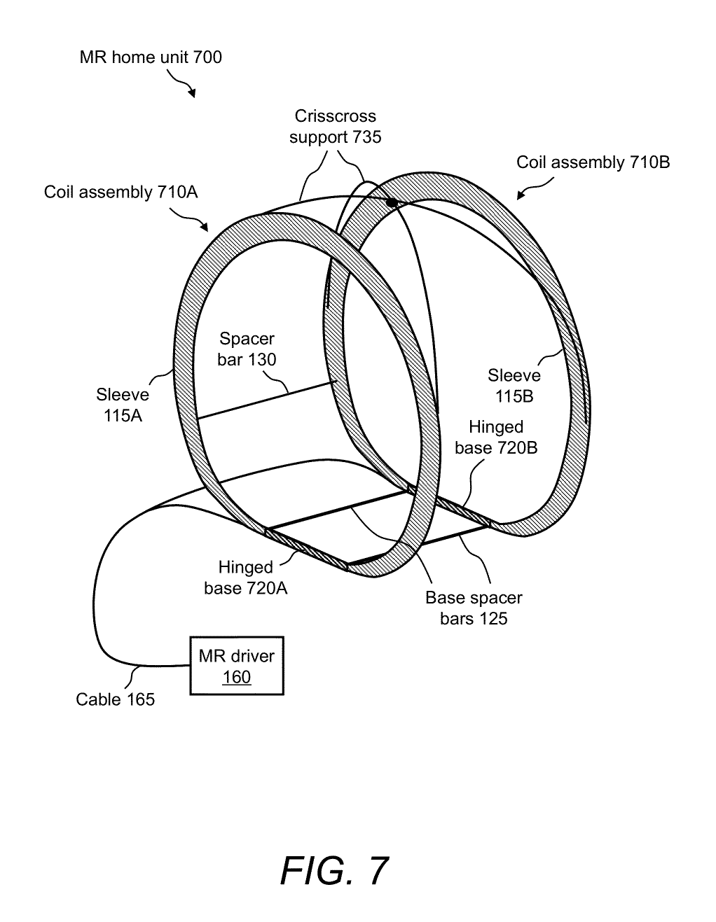

FIG. 7 comprises another embodiment of a magnetic resonance home unit according to the present disclosure. FIG. 7, illustrates an isometric view of an MR home unit 700, which is another example embodiment of an MR home unit device. MR home unit 700 is similar to the MR home unit 100 described with regard to FIG. 1. But in the embodiment shown in FIG. 7, the collapsible or hinged bases are modified to allow MR home unit 700 to have a different appearance than MR home unit 100. For example, when viewed from the side, MR home unit 100 may comprise an arch-shaped appearance whereas MR home unit 700 may comprise a more circular appearance.

As shown in FIG. 7, MR home unit 700 may comprise a pair of foldable coil assemblies 710. In the embodiment shown in FIG. 7, these coil assemblies comprise a coil assembly 710A and a coil assembly 710B. In the embodiment shown in FIG. 7, coil assemblies 710A and 710B are enclosed in sleeves 115A, 115B, respectively. In the embodiment shown in FIG. 7, the coil assemblies 710 are mounted on hinged bases 720, which are foldable. For example, coil assembly 710A is mounted on hinged base 720A and coil assembly 710B is mounted on hinged base 720B. In some embodiments, coil assemblies 710A and 710B may be substantially the same as coil assemblies 110A, 110B of MR home unit 100 except that they comprise hinged bases 720 instead of hinged bases 120.

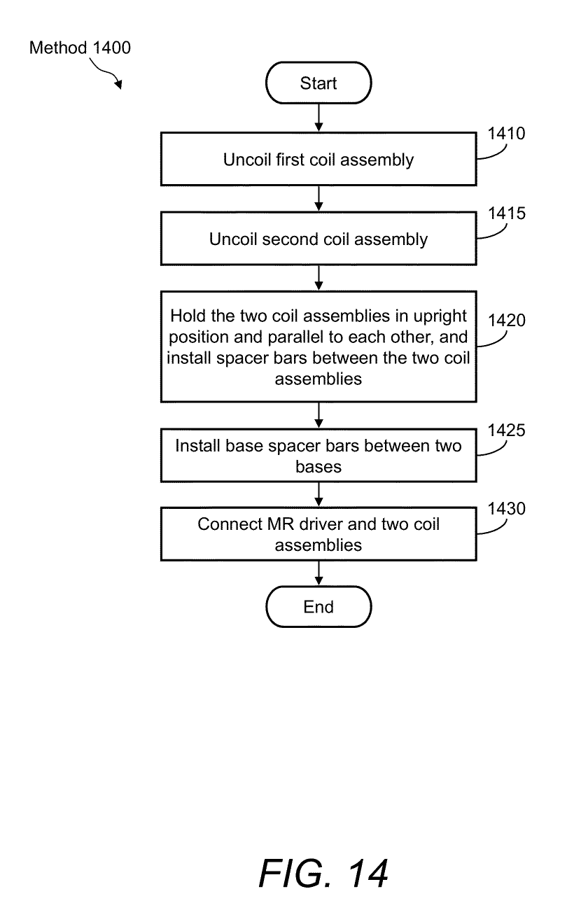

Further, in some embodiments, MR home unit 700 may comprise a different arrangement of spacer bars as compared with MR home unit 100. For example, MR home unit 700 may comprise the two base spacer bars 125 between hinged bases 720A and 710B, only one spacer bar 130 between sleeve 115A, 115B, and a crisscross support 735. In this example, the ends of the two rods that form the crisscross support 735 may be, for example, fitted into pockets that are provided in sleeves 115A, 115B. Further, MR home unit 700 may be assembled according to method 1400 of FIG. 14, with the additional step of installing the crisscross support 835 between sleeves 115A, 115B.

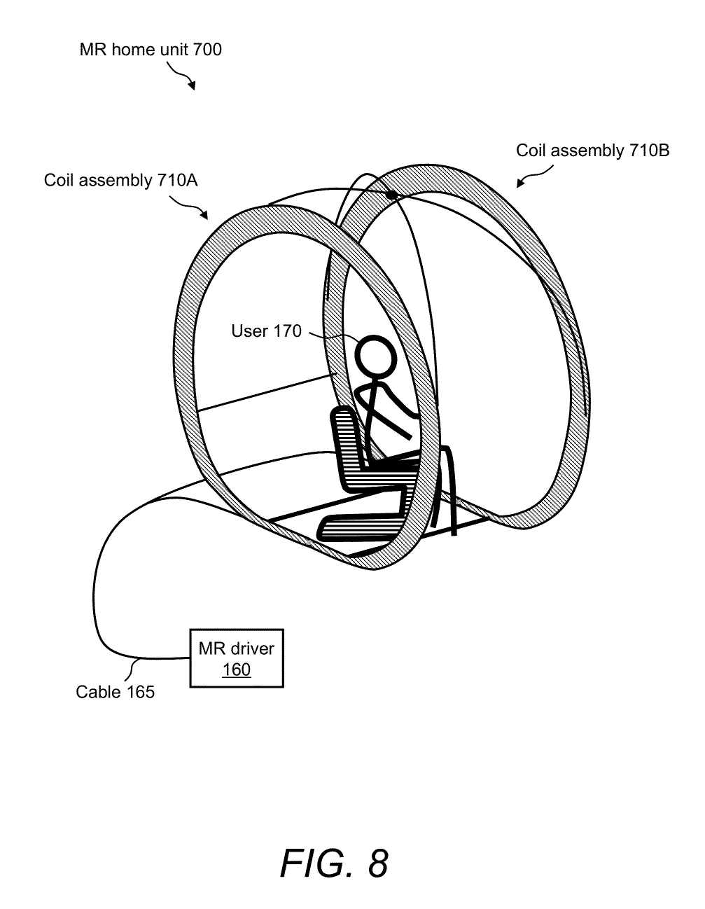

FIG. 8 illustrates a view of MR home unit 700 of FIG. 7 with user 170 sitting therein, wherein user 170 is the subject of an MR treatment. As discussed above with regard to FIG. 1, in some embodiments, the MR treatment may be applied to a different subject, e.g., a non-human subject, such as an animal (dog, cat, lizard, bird, fish) a plant (e.g., a houseplant or crop), or some inanimate object (e.g., water or building materials).

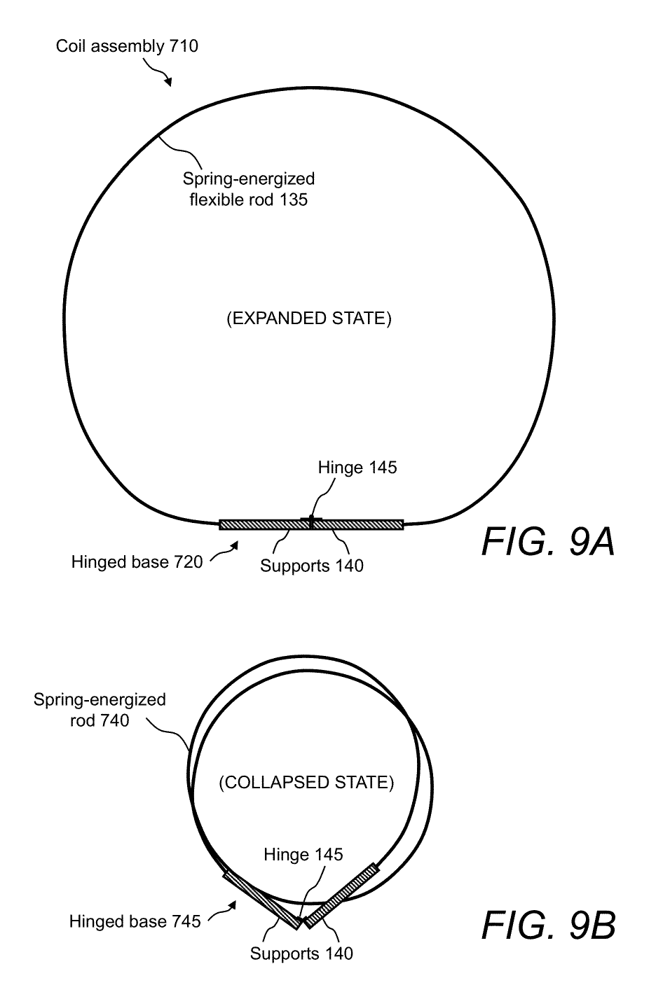

Turning now to FIGS. 9A and 9B, FIGS. 9A and 9B, show illustrations of an expanded state and a collapsed state of a magnetic resonance home unit according to one embodiment of the present disclosure. As shown in FIGS. 9A and 9B illustrate side views of spring-energized flexible rod 135 and hinged base 720 of MR home unit 700 of FIG. 7. In some embodiments, hinged base 720 is substantially the same as hinged base 120 except that, (1) in some embodiments, supports 140 of hinged base 720 are shorter than supports 140 of hinged base 120, (2) in some embodiments, hinge 145 of hinged base 720 is installed on the top side of hinged base 720, whereas in hinged base 120 the hinge 145 is installed on the bottom side, and (3) in some embodiments, the ends of spring-energized flexible rod 135 are installed into the ends of supports 140 of hinged base 720 (in the plane of supports 140), whereas in some embodiments, in hinged base 120 the ends of spring-energized flexible rod 135 are installed perpendicular to the supports 140. In some embodiments, no hinge is necessary, e.g., hinged base 720 may comprise a flexible base or a fixed base. In other embodiments, hinged base 720 is not necessary, and the device relies on other means for structural support, e.g., one or more supports coupled to or between the two coil assemblies. In some embodiments, the length of supports 140 of hinged base 720 are from about 12 inches to about 24 inches in one example, or about 18 inches in another example.

FIG. 9A shows spring-energized flexible rod 135 in a fully expanded state and with hinge 145 locked. Further, FIG. 9B shows spring-energized flexible rod 135 in a collapsed state. For example, FIG. 9B shows that hinge 145 can be unlocked, which allows spring-energized flexible rod 135 to be folded into a small form factor. In the embodiment shown in FIG. 9B, hinge 145 is shown unlocked, which allows the two supports 140 to fold such that hinge 145 can move away from the loop. At the same time, spring-energized flexible rod 135 can be twisted into a figure-eight, then the two loops of the figure-eight can be folding over one another to create the small form factor that is shown in FIG. 9B. For purpose of illustration, FIGS. 9A and 9B show only the spring-energized flexible rod 135 portion of the coil assembly 710. However, it is the intent that each fully assembled coil assembly 710 be collapsible, a feature that is enabled by the inclusion of spring-energized flexible rod 135. In some embodiments, not shown in FIG. 9B, the flexible rod 135 could be folded a plurality of times to form a smaller package. Further, in some embodiments, other components may fold in one or more directions to lead to an even small folded package.

MR home units 100 and 700 are not limited to the specific arrangements of hinged bases, spacer bars, base spacer bars, and/or crisscross supports that are shown and described with reference to FIGS. 1 through 9B. In some embodiments, MR home units 100 and 700 can use other mechanisms to allow MR home units 100 and 700 to be physically supported, lightweight, inexpensive, easily assembled, and easily disassembled.

In the embodiments described above, magnetic resonance home units are not limited to home use only. MR home units can be used in any clinical or non-clinical setting. For example, in addition to being used in homes, MR home units can be used in athletic environments and can travel with athletic teams because of their portability. Furthermore, in some embodiments, MR home units can be used in any environment in which it is beneficial to have a simple and affordable MR device, e.g., offices, spas, locker rooms, training facilities, gyms, hotels, motels, apartments, etc.

Further, magnetic resonance home units may be distinguishable from other device for applying a magnetic resonance treatment, because one or more components of a home unit may comprise the capability to be folded into a smaller form factor, e.g., for shipping or for travel. Further, in some embodiments, a magnetic resonance home unit may not comprise a complete fiberglass shell as may be found on a clinical unit. In addition, a magnetic resonance home unit may comprise a plurality of components produced from lightweight and flexible materials. This leads to substantially cheaper production and greater portability. Further, a magnetic resonance home unit may be configured to operate based on signals received from a home computing device, e.g., a home computer or tablet executing magnetic resonance software. Thus, in some embodiments, a magnetic resonance home unit may comprise a less sophisticated user interface than a clinical unit (or, in some embodiments, no user interface). Similarly, in some embodiments, a magnetic resonance home unit may be configured to receive treatment information from a remote database. In some embodiments, this remote database may comprise treatment data associated with a patient or subject or treatment data associated with like situated patients or subjects, from which a treatment protocol for the patient may be developed. In some embodiments, software associated with a magnetic resonance home unit, or associated with a computer that may be coupled to the magnetic resonance home unit, may be configured to receive this treatment protocol, and control a driver associated with the magnetic resonance home unit to output a magnetic resonance to the patient or subject.

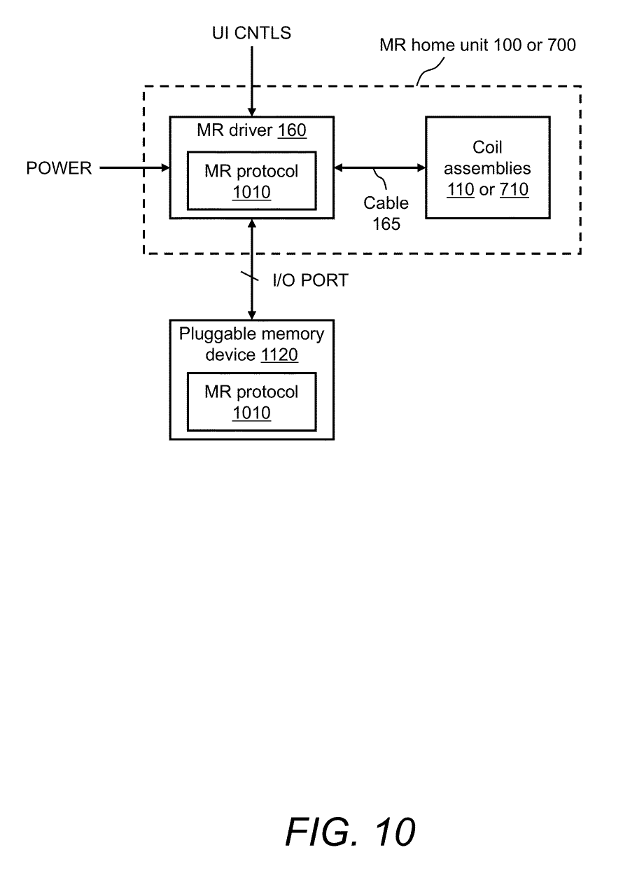

Turning now to FIG. 10, FIG. 10 illustrates a block diagram of a magnetic resonance home unit according to one embodiment. As shown in FIG. 10, MR driver 160 is connected to coil assemblies 110 or 710 described above. In one embodiment, MR driver 160, includes a power input port (POWER), an input/output port (I/O PORT), and one or more user interface controls (UI CNTLS).

In one embodiment, POWER can be supplied by a standard 120-volt, 60 Hz, AC outlet. In some embodiments, MR driver 160 can include a power conditioning function (not shown) to process the AC input in a manner to satisfy the specific AC and/or DC power requirements a magnetic resonance home unit. In other embodiments, other types of power supplies may be used, e.g., higher or lower voltage or frequency AC power supplies, or a DC power supply.

I/O PORT can be any type of I/O port capable of transferring digital information in and out of MR driver 160. In some embodiments, I/O PORT may be configured to load an MR protocol 1010 into MR driver 160. In some embodiments, MR protocol 1010 may comprise, for example, a user-specific MR protocol, such as the user-specific MR protocol for user 170. In some embodiments, MR protocol 1010 comprises one or more of the specific magnetic waveform parameters, for example, waveform type (e.g., sinusoidal, rectilinear, square), amplitude (e.g., 1 mV), and frequency (e.g., 10 Hz) of the uniform homogeneous magnetic field that is generated between the two coil assemblies. Information in MR protocol 1010 may also specify the duration of the MR treatment. In some embodiments, I/O PORT can be one or more ports for receiving a pluggable memory device 1020. Pluggable memory device 1020 can be, for example, a pluggable USB-based device, a pluggable secure digital (SD) card, and the like. Accordingly, in some embodiments, I/O PORT can support, for example, a USB-based device and/or an SD card. In other embodiments, I/O PORT may comprise for example, a network interface, a wireless interface, serial port, or a user interface, such as a keyboard, mouse, touch-screen, button, or some other type of user interface or data port.

UI CNTLS are any controls by which user 170 can operate and/or monitor a MR home units 100, 700. In some embodiments, UI CNTLS can include, for example, a power on/off button, a treatment start/stop button, and a treatment pause/resume button, as well as various visual indicators, such as one or more light-emitting diodes (LEDs). In one example, UI CNTLS includes a “power on” indicator, a “ready” indicator, a “running” indicator, and a “paused” indicator.

In some embodiments, MR protocol 1010 can be supplied to MR driver 160 via pluggable memory device 1120. Pluggable memory device may comprise a USB drive, a smartphone, a network drive, a wireless or wired network interface, or some other type of wired or wireless connection to a data store. In such an embodiment, MR home units 100, 700 may operate based on information in MR protocol 1010. Further, in such an embodiment, user 170 may plug pluggable memory device 1020 into I/O PORT of MR driver 160. In such an embodiment, a control module (not shown) of MR driver 160 automatically detects the presence of pluggable memory device 1020 in I/O PORT and then automatically reads in and stores a local copy of MR protocol 1010. In such an embodiment, pluggable memory device 1020 can then be removed from I/O PORT. In one embodiment, instead of storing a local copy of MR protocol 1110 on MR driver 160, pluggable memory device 1020 may remain plugged into I/O PORT so that MR driver 160 can access MR protocol 1010 while performing the MR treatment. In some embodiments, once MR driver 160 has acquired the MR protocol 1010, the “ready” indicator turns on and MR home unit 100 or 700 is ready for use, wherein MR home unit 100 and 700 can operate based on information in MR protocol 1010.

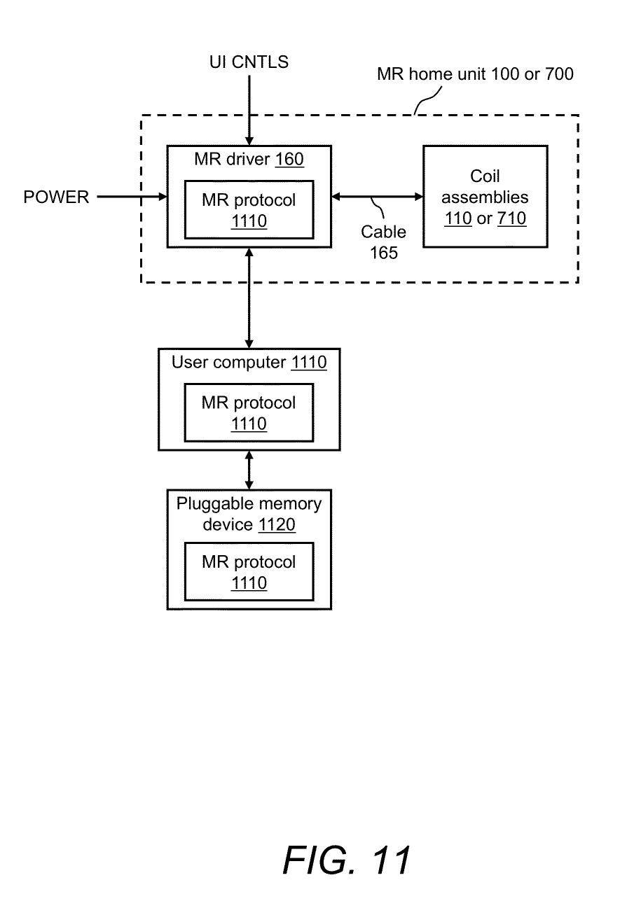

Turning to FIG. 11, FIG. 11 shows another block diagram of a magnetic resonance home unit according to one embodiment. As shown in FIG. 11 MR home units 100, 700 may operate in a configuration that includes a user computer 1110. In some embodiments, user computer 1110 can be any computing device, such as, but not limited to, a desktop computer, a laptop computer, a tablet computer, a mobile phone, a smart phone, a PDA device, and the like. User computer 1110 can be connected to MR driver 160 in any wired or wireless fashion. Accordingly, in this example, I/O PORT additionally supports standard wired and wireless communication mechanisms. For example, I/O PORT can support standard wired USB and Ethernet connections, as well as a standard WiFi or Bluetooth wireless connection. In some embodiments POWER can be supplied using Power-over-Ethernet (PoE) rather than using a standard AC outlet or via a battery or other type of DC or AC power connection.

Further, in some embodiments, MR protocol 1110 can be supplied to user computer 1110 via pluggable memory device 1120. Then, MR protocol 1110 is transferred from user computer 1110 to MR driver 160. In some embodiments, once MR driver 160 has acquired the MR protocol 1110, the “ready” indicator turns on and MR home unit 100 or 700 is ready for use, wherein MR home unit 100 and 700 can operate based on information in MR protocol 1110.

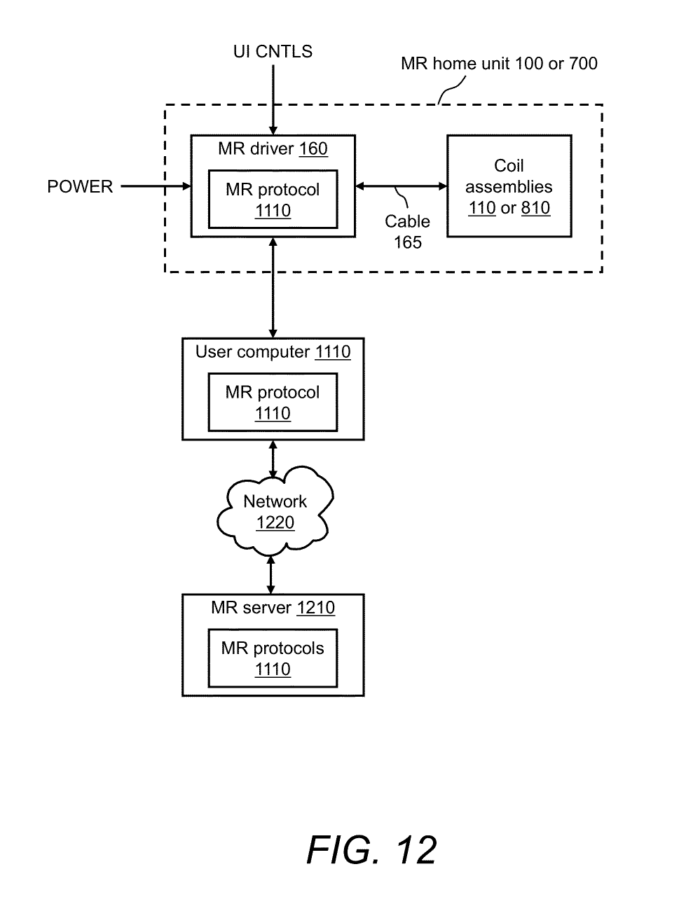

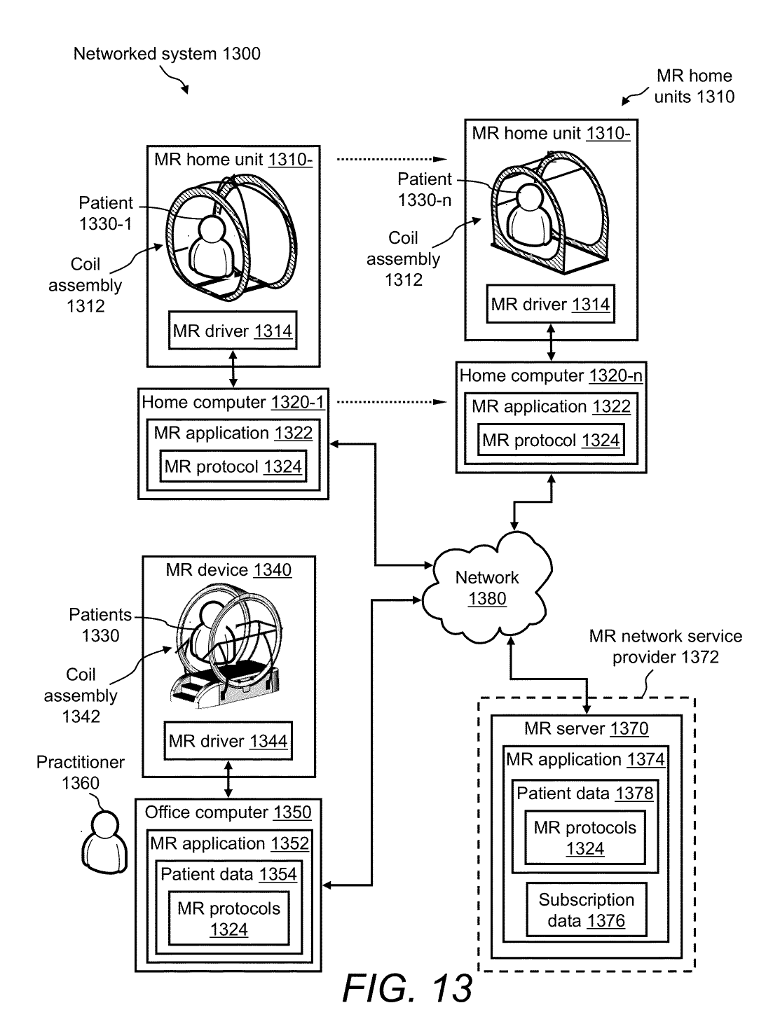

Turning now to FIG. 12, FIG. 12 shows another block diagram of a magnetic resonance home unit according to one embodiment. As shown in FIG. 13 MR home units 100 and 700 may operate in a configuration that includes user computer 1110 that is networked. For example, user computer 1110 is connected to an MR server 1210, which can be any centralized server that can be accessed by one or more user computers 1110 belonging to one or more users 170, respectively. For example, user computer 1110 can access MR server 1210 via a network 1220. In some embodiments, network 1220 is, for example, any standard wired or wireless wide area network (WAN) or local area network (LAN), a Bluetooth connection, a cellular data connection, a telephone connection, a cable connection, a satellite data connection, or some other type of data connection. User computer 1110 and MR server 1210 are connected to network 1220 by any wired or wireless means.

In some embodiments, user 170 uses his/her user computer 1110 to retrieve his/her user-specific MR protocol 1110 from MR server 1210. Then, MR protocol 1110 is transferred from user computer 1110 to MR driver 160. In some embodiments, once MR driver 160 has acquired the MR protocol 1110, the “ready” indicator turns on and MR home unit 100 or 700 is ready for use, wherein MR home unit 100 and 800 can operate based on information in MR protocol 1110.

In the embodiments shown in FIGS. 10-12, MR driver 160 may acquire the user-specific MR protocol 1110. For example, in FIG. 11, MR driver 160 acquires the user-specific MR protocol 1110 via pluggable memory device 1120. In FIG. 11, MR driver 160 acquires the user-specific MR protocol 1110 via user computer 1110. In FIG. 12, MR driver 160 acquires the user-specific MR protocol 1110 via user computer 1110 and MR server 1210. In some embodiments, user 170 can then manually press the start/stop button on MR driver 160 or use an MR control application at user computer 1110 to initiate the MR treatment. When the MR treatment is initiated, the “running” indicator turns on. Upon initiating the MR treatment, coil assemblies 110A, 110B of MR home unit 100 or coil assemblies 710A, 710B of MR home unit 700 are energized and a substantially uniform homogeneous magnetic field is generated between the two coils. Coil assemblies 110A, 110B of MR home unit 100 or coil assemblies 710A, 710B of MR home unit 700 are automatically deactivated when the duration of the MR treatment per MR protocol 1110 is reached. Upon deactivation, the “running” indicator on MR driver 160 turns off.

Turning now to FIG. 13, shows another block diagram of a magnetic resonance home unit according to one embodiment. FIG. 13, illustrates a block diagram of one embodiment of a networked system 1300 for supporting a plurality of MR home units 1310. In some embodiments, networked system 1300 comprise at least one MR device 1340 that is operated by at least one practitioner 1360 in a clinical setting. Networked system 1300 may further comprise the plurality of MR home units 1310 that are operated by a plurality of patients 1330 of practitioner 1360. Networked system 1300 may further comprise a centralized MR server 1370 that is accessible via a network 1380 by both practitioner 1360 for operating his/her MR device 1340 and patients 1330 for operating their respective MR home units 110. While networked system 1300 shows one practitioner 1360 supporting a plurality of patients 1330, networked system 1300 is not limited to only one practitioner 1360. In some embodiments, networked system 1300 can support any number of practitioners 1360, wherein each practitioner 1360 can support any number of the same or different patients 1330.

In some embodiments, networked system 1300 can include any number of MR home units 1310, such as MR home units 1310-1 through 1310-n. Accordingly, patients 1330-1 through 1330-n are associated with MR home units 1310-1 through 1310-n, respectively. In some embodiments, networked system 1300 also includes a home computer 1320 for each MR home unit 1310. Accordingly, home computers 1320-1 through 1320-n are associated with MR home units 1310-1 through 1310-n. Home computers 1320 are connected to network 1380 for communicating with MR server 1370.

In some embodiments, each MR home unit 1310 includes a coil assembly 1312 and an MR driver 1314 that is configured for home use. In some embodiments, each MR home unit 1310 is a home use MR device for whole-body immersion into precisely tuned, low-level electromagnetic fields for enhanced relaxation, comprising one or more of the features discussed above with regard to FIGS. 1-12. In other embodiments, the MR home unit may comprise a home use MR device for immersion of body parts (e.g., arms, legs, hands, feet, knees, elbows, wrists, neck, head, etc.) into precisely tuned, low-level electromagnetic fields.

In some embodiments, the settings of MR driver 1314 may be determined by a patient-specific MR treatment protocol, such as an MR protocol 1324. For example, in one embodiment, MR protocol 1324, which contains patient-specific information, resides on the home computer 1320 that is connected to the MR driver 1314 of each MR home unit 1310. In some embodiments, an MR application 1322 on home computer 1320 can be used to transfer the information of MR protocol 1324 to MR driver 1314. In some embodiments, MR application 1322 can be, for example, a web-based application that is accessible via network 1380 or a software application that is installed and running locally on home computer 1320. In some embodiments, using MR application 1322 and network 1380, the patient 1330 can login to MR server 1370 and access his/her own unique MR protocol 1324 that is predetermined and preapproved by practitioner 1360.

In some embodiments, for each patient 1330, practitioner 1360 determines the settings of MR driver 1344 based on positive results of the MR treatment in MR device 1340. In so doing, practitioner 1360 may determine the MR protocol 1324 for each patient 1330. In some embodiments, using an office computer 1350, practitioner 1360 may use an MR application 1352 to enter the parameters of the MR protocol for controlling MR device 1340. MR application 1352 can be, for example, a web-based application that is accessible via network 1380 or a software application that is installed and running locally on office computer 1350.

In some embodiments, once practitioner 1360 determines the correct parameters for a given patient 1330, the parameters may be logged in the patient 1330's MR protocol 1324. For example, in some embodiments, MR application 1352 at office computer 1350 is used to manage patient data 1354. In some embodiments, patient data 1354 includes the records of all patients 1330 associated with networked system 1300, which includes the unique MR protocols 1324 for each of the patients 1330.

Further, in some embodiments, practitioner 1360 may be authorized to upload patient data 1354, which includes MR protocols 1324, to an MR server 1370. In this way, the MR protocols 1324 stored at MR server 1370 are MR protocols that have been predetermined and preapproved by practitioner 1360. However, these MR protocols 1324 at MR server 1370 are now accessible by patients 1330 for use at home on their MR home units 1310.

Home computers 1320 and office computer 1350 can be any computing device, such as, but not limited to, a desktop computer, a laptop computer, a tablet computer, a mobile phone, a smart phone, a PDA device, and the like. MR server 1370 can be any centralized server (including a cloud server) that can be accessed by home computers 1320 belonging to patients 1330 and by office computer 1350 belonging to practitioner 1360. Home computers 1320, office computer 1350, and MR server 1370 are connected to network 1380 by any wired or wireless means. Network 1380 can be a wide area network (WAN) or a local area network (LAN) for connecting to the Internet.

In some embodiments, MR server 1370 and networked system 1300 are owned and operated by an MR network service provider 1372, who controls access to all entities of networked system 1300. In some embodiments, an MR application 1374 at MR server 1370 is used to access and manage patient data 1378, which comprises the unique information for each patient 1330 include the unique MR protocols 1324. Additionally, because, in some embodiments, access to networked system 1300 can be provided as a service for a fee, subscription data 1376 resides at MR server 1370. Accordingly, in some embodiments, MR application 1374 may be also used to manage subscriptions to networked system 1300.

MR application 1374 can be, for example, a web-based application that is accessible via network 1380 or a software application that is installed and running locally on MR server 1370. In some embodiments, MR application 1374 is designed to manage any and all aspects of networked system 1300. For example, in on embodiment, only authorized personnel (e.g., system administrators, managers, etc) of networked system 1300 are authorized to use MR application 1374 to manage the content stored at MR server 1370 and to manage access to MR server 1370. In other embodiments, the function of MR application 1352 at office computer 1350 is limited to only those functions to which practitioner 1360 are allowed. Similarly, in some embodiments, MR application 1322 at home computers 1320 is limited to only those functions to which patients 1330 are allowed.

In one embodiment a patient 1330 may travel to the location (e.g., office or clinic) of practitioner 1340 to receive MR treatments for the purpose of determining an MR treatment protocol that is effective for treating pain and/or relieving symptoms of the patient 1330. In such an embodiment, under the guidance of practitioner 1340, patient 1330 receives MR treatments using MR device 1340 and over time (e.g., multiple visits) an effective MR treatment protocol can be determined. Further, in such an embodiment, once an effective MR treatment protocol is determined, practitioner 1360 stores the information in a unique MR protocol 1324 for that patient 1330. Then, the unique MR protocol 1324 for the patient 1330 may be uploaded from office computer 1350 to MR server 1370.

Furthermore, in such an embodiment, the unique MR protocol 1324 for the patient 1330 may then be accessible using home computer 1320 and available for use with his/her MR home unit 1310. As a result, networked system 1300 supports an environment in which patients 1330 can conveniently operate and treat themselves at home using MR home units 1310, reducing or substantially eliminating the need to travel to or make appointments with their practitioner 1360.

### Illustrative Method for a Magnetic Resonance Home Unit

Referring now to FIGS. 14-16, which comprises a flow charts describing an exemplary embodiment for methods for a magnetic resonance home unit. In some embodiments, the stages in FIGS. 14-16 may be implemented in program code that is executed by a processor, for example, the processor in a general purpose computer, a mobile device, or server. In some embodiments, these stages may be implemented by a group of processors, for example, a processor on a mobile device and processors on one or more general purpose computers, such as servers. Further, although the operations shown in FIGS. 14-16 are described as sequential processes, many of the operations can be performed in parallel or concurrently. In addition, the order of the operations may be rearranged. Similarly, the processes shown in FIGS. 14-16 may comprise additional steps not included in the Figures.

As shown in FIG. 14, the method 1400 of assembling an MR home unit, which may be performed, for example, by user 170. The method 1400 begins at a step 1410, the first coil assembly is uncoiled or unfolded. In some embodiments, a hinged or flexible based may then be locked. For example, coil assembly 110A is provided in the collapsed state, as shown in FIG. 3B. User 170 uncoils coil assembly 110A into the fully expanded, as shown in FIG. 3A and FIG. 5, and then locks hinged base 120. User 170 then sets coil assembly 110A aside.

At a step 1415, the second coil assembly is uncoiled or unfolded. In some embodiments, a hinged or flexible based may then be locked. For example, coil assembly 110B is provided in the collapsed state, as shown in FIG. 3B. User 170 uncoils coil assembly 110B into the fully expanded, as shown in FIG. 3A and FIG. 5, and then locks hinged base 120.

At a step 1420, with the hinged bases on the floor, the two coil assemblies are held in the upright position and parallel to each other. Then, the spacer bars are installed between the two coil assemblies. For example, with hinged bases 120A, 120B on the floor, user 170 holds coil assemblies 110A, 110B in an upright position and parallel to each other. User 170 then installs the spacer bars 130 between coil assemblies 110A, 110B. For example, one-by-one the ends of each of the spacer bars 130 is mechanically affixed to sleeves 115A, 115B using a set of fasteners 610, as shown in FIG. 6.

At a step 1425, the base spacer bars are installed between the two hinged bases. For example, user 170 installs two or more base spacer bars 125 between hinged bases 120A, 120B of coil assemblies 110A, 110B, respectively. In some embodiments, one-by-one the ends of each of the base spacer bars 125 are mechanically affixed to hinged bases 120A, 120B. In one example, base spacer bars 125 are affixed to hinged bases 120A, 120B using screws or nuts and bolts.

At a step 1430, the cable is mechanically and electrically connected between the MR driver and the two coil assemblies. For example, user 170 plugs one end of cable 165 into MR driver 160 and the other end of cable 165 into coil assemblies 110A, 110B. In other embodiments, a different type of connection may be made between the MR driver 160 and coil assemblies 110A, 110B. For example, in some embodiments, the connection may comprise a wireless connection such as by Bluetooth, wi-fi, or some other type of connection.

In some embodiments, to disassemble MR home unit 100, user 170 performs substantially the reverse of the operations described in method 1400. The ease of assembly and disassembly lends well to the portability of MR home unit 100. Further, in some embodiments, the entire system may be able to fit into a single, relatively small box, which may be moved by one person. In some embodiments, the coils may arrive to the user fully assembled and require that they only be connected to each other and the MR Driver. Further, upon arrival, the MR driver may be connected to a computer, tablet, smartphone, or server, from which a database of treatment data or treatment protocols may be accessed.

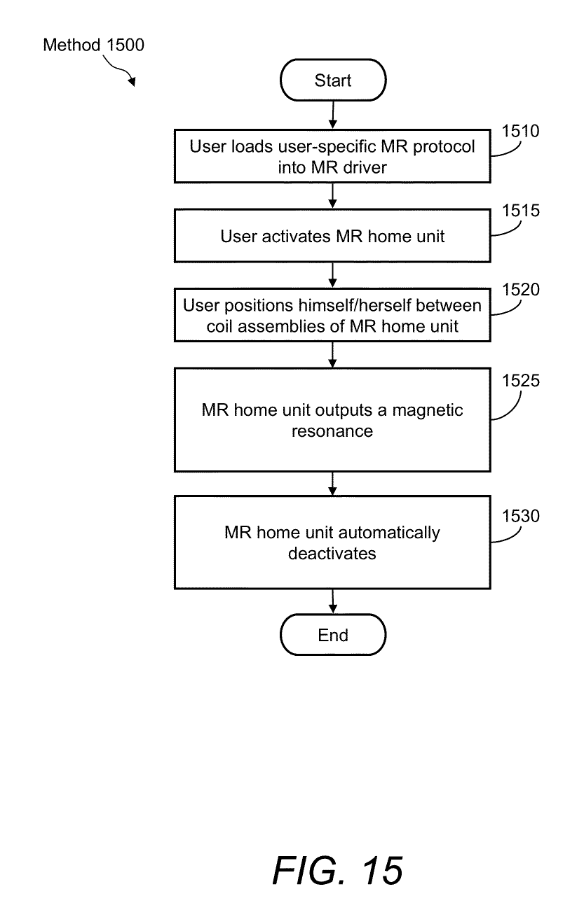

FIG. 15 illustrates a flow diagram of an example of a method 1500 of using an MR home unit. Using method 1500, user 170 can operate MR home unit 100 or 700 at his/her convenience. The method 1500 begins at a step 1510, when user 170 loads his/her user-specific MR protocol 1110 into MR driver 160 of a MR home unit. For example, in one embodiment, a user 170 may use a pluggable memory device 1120 to load his/her user-specific MR protocol 1110 into MR driver 160. In another embodiment, user 170 may use a user computer 1110 to load his/her user-specific MR protocol 1110 into MR driver 160. In another embodiment, user 170 may retrieves his/her user-specific MR protocol 1110 from MR server 1210 and then uses user computer 1110 to load the MR protocol 1110 into MR driver 160.

At a step 1515, user 170 activates an MR home unit. In one embodiment, example, user 170 may manually press the start/stop button on MR driver 160 to activate the MR home unit. In some embodiments, when the MR treatment is initiated, the “running” indicator turns on. In another embodiment, user 170 may use an MR control application at user computer 1110 to initiate the MR treatment. In some embodiments, when the MR treatment is initiated, the “running” indicator of the MR control application is activated.

At a step 1520, user 170 positions himself/herself between coil assemblies of the MR home unit. For example, user 170 can stand or sit between the two coil assemblies. In some embodiments, the user may only position some part of his or her body between the coil assemblies (e.g., only the user's arm(s), leg(s), or one or more joint(s)).

At a step 1525, the MR home unit outputs magnetic resonance according to the MR protocol 1110. In one example, the duration of the MR treatment per MR protocol 1110 is 60 minutes. The user is not required to stay in the MR home unit for the prescribed amount of time, but may not receive the full benefits of the treatment when outside of the magnetic field.

At a step 1530, MR home unit may automatically deactivate and user 170 exits the MR home unit. For example, at end of prescribed amount of time, e.g., 60 minutes, or some other length of time.

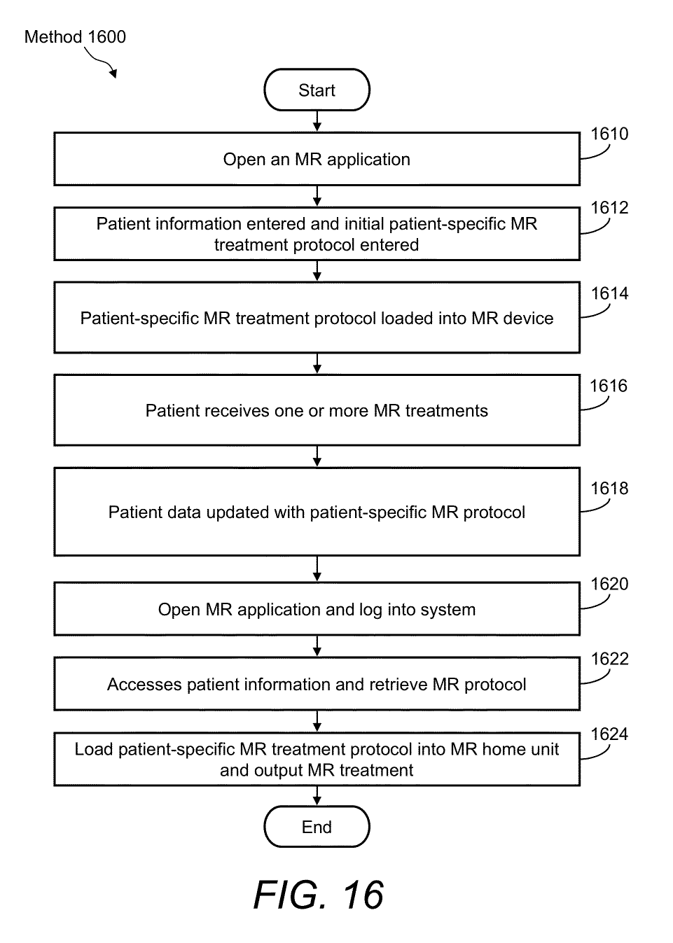

FIG. 16 illustrates a flow diagram of an example of a method 1600 of using networked system 1300 of FIG. 13. The method 16 begins at step 1610, when an MR application 1352 on office computer 1350, or other computing device, such as a mobile device, tablet computer, smartphone, dedicated microcontroller, or server is opened and logged into a networked system 1300.

At a step 1612, patient information of a specific patient 1330 is entered and an initial patient-specific MR treatment protocol (e.g., MR protocol 1324) is entered. For example, practitioner 1360 uses MR application 1352 to specify specific magnetic waveform parameters, such as waveform type (e.g., sinusoidal, rectilinear, square, saw-tooth, white noise, pink noise), amplitude (e.g., 1 mV, 2 mV, or another greater or lesser value), frequency (e.g., 10 Hz, 20 Hz, 30 Hz, or another frequency), and time duration of the uniform homogeneous magnetic field that is generated by MR device 1340. In this way, an initial MR protocol 1324 is created for the specific patient 1330.

In other embodiments, a patient 1330 may create his or her own treatment protocol, for example, through trial and error, through adjusting specific settings, or by selecting one of a plurality of treatment options or predesigned MR treatment protocols. In other embodiment, the MR treatment protocol may not be specially designed for a specific patient. For example, in some embodiments, the treatment protocol may be designed for treatment of a specific ailment, body part, or for a particular subject. Thus, in some embodiments, rather than a patient-specific MR treatment protocol, the treatment protocol may comprise one of a plurality of possible general MR treatment protocols.

At a step 1614, patient-specific MR treatment protocol loaded into MR device 1340. Further, in some embodiments, patient 1330 may receive one or more MR treatments according to initial MR protocol 1324. In some embodiments, this step may involve several visits to practitioner 1360.

At a step 1616, over time, patient 1330 receives multiple MR treatments and the MR treatment protocol is refined. In some embodiments, the practitioner and or patient may establish an MR protocol 1324 that is suitable for use with MR home unit 1310. For example, practitioner 1360 may use MR application 1352 to adjust the specific magnetic waveform parameters, such as waveform type (e.g., sinusoidal, rectilinear, square, saw-tooth, white noise, pink noise), amplitude (e.g., 1 mV, 2 mV, or another greater or lesser value), frequency (e.g., 10 Hz, 20 Hz, 30 Hz, or another frequency), and/or time duration of the uniform homogeneous magnetic field that is generated by MR device 1340 until a suitable MR protocol 1324 is determined. In some embodiments, the patient may receive only one treatment. In other embodiments, the patient may receive a plurality of different treatments for a plurality of different body parts, e.g., treatments for one or more of the patient's arms, legs, joints, etc.

At a step 1618, patient data 1354 is updated with patient-specific MR protocol 124. For example, in some embodiments, practitioner 1360 may upload the current patient data 1354 to an MR server 1370. In so doing, patient data 1378 at MR server 1370 may be updated with the approved patient-specific MR protocol 1324; making the preapproved MR protocol 1324 accessible from the patient 1330's home for use with his/her MR home unit 1310.

At a step 1620, patient 1330 opens MR application 1322 at his/her home computer 1320 and logs into networked system 1300. In some embodiments, networked system 1300 may be remote from the user. In other embodiments, networked system may be associated with the user's location, e.g., accessed via a local area network (LAN) or a wireless area network (WAN). In still other embodiments, the networked system may comprise a system with network capability, but no network access.

At a step 1622, patient 1330 accesses his/her patient information in patent data 1378 at MR server 1370 and retrieves his/her preapproved MR protocol 1324. In some embodiments, the patient may retrieve the MR protocol via the network connection from a remote database. In some embodiments, the patient may retrieve the MR protocol from a local data store, such as a local hard drive or a local external drive, e.g., a USB drive or a drive associated with a mobile device. In still other embodiment, the patient may retrieve the MR protocol via a wireless connection to either a local or remote data store.

At a step 1624, patient 1330 loads his/her preapproved MR protocol 1324 into MR home unit 1310 and receives an MR treatment according to the preapproved MR protocol 1324. In some embodiments, preapproved may mean that a provider has designed and/or prescribed the treatment protocol. In other embodiments, preapproved may mean that the patient has selected the treatment protocol. In still other embodiments, preapproved may mean that the magnetic resonance home unit is capable of outputting a magnetic resonance treatment according to the protocol.

In the embodiments described above, magnetic resonance home units are not limited to home use only. MR home units can be used in any clinical or non-clinical setting. For example, in addition to being used in homes, MR home units can be used in athletic environments and can travel with athletic teams because of their portability. Furthermore, in some embodiments, MR home units can be used in any environment in which it is beneficial to have a simple and affordable MR device, e.g., offices, spas, locker rooms, training facilities, gyms, hotels, motels, apartments, etc.

### Advantages of a Magnetic Resonance Home Unit

Systems and methods for a magnetic resonance home unit may provide numerous advantages. First because the MR home unit is in the patient's home, the MR home unit is easily accessible. As a result, patients can conveniently receive their MR treatments at home at any time and with any frequency. Further, an MR home unit is designed to be collapsible and lightweight for ease of portability and/or shipment. Thus, a user may easily ship or travel with an MR home unit, and therefore be less likely to miss treatments.

Further, in some embodiments, MR treatments that are performed using an MR home unit are based on user-specific treatment protocols that are predetermined and preauthorized by the patient's service provider. This may enable the patient to receive a specifically designed treatment, but from the comfort of the user's own home. Moreover, systems and methods according to the present disclosure make it possible for a user to access or create personalized treatment protocols via a networked computer, and thus keep the user from having to repeatedly travel to a service provider to receive a MR treatment.

### General Considerations

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

Having described several example configurations, various modifications, alternative constructions, and equivalents may be used without departing from the spirit of the disclosure. For example, the above elements may be components of a larger system, wherein other rules may take precedence over or otherwise modify the application of the disclosure. Also, a number of steps may be undertaken before, during, or after the above elements are considered. Accordingly, the above description does not bound the scope of the claims.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

Embodiments in accordance with aspects of the present subject matter can be implemented in digital electronic circuitry, in computer hardware, firmware, software, or in combinations of the preceding. In one embodiment, a computer may comprise a processor or processors. The processor comprises or has access to a computer-readable medium, such as a random access memory (RAM) coupled to the processor. The processor executes computer-executable program instructions stored in memory, such as executing one or more computer programs including a sensor sampling routine, selection routines, and other routines to perform the methods described above.

Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example tangible computer-readable media, that may store instructions that, when executed by the processor, can cause the processor to perform the steps described herein as carried out, or assisted, by a processor. Embodiments of computer-readable media may comprise, but are not limited to, all electronic, optical, magnetic, or other storage devices capable of providing a processor, such as the processor in a web server, with computer-readable instructions. Other examples of media comprise, but are not limited to, a floppy disk, CD-ROM, magnetic disk, memory chip, ROM, RAM, ASIC, configured processor, all optical media, all magnetic tape or other magnetic media, or any other medium from which a computer processor can read. Also, various other devices may include computer-readable media, such as a router, private or public network, or other transmission device. The processor, and the processing, described may be in one or more structures, and may be dispersed through one or more structures. The processor may comprise code for carrying out one or more of the methods (or parts of methods) described herein.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, it should be understood that the present disclosure has been presented for purposes of example rather than limitation, and does not preclude inclusion of such modifications, variations and/or additions to the present subject matter as would be readily apparent to one of ordinary skill in the art.

What is claimed is:

1. A method for a home magnetic resonance treatment comprising:
opening a magnetic resonance application;
accessing a magnetic resonance treatment protocol associated with a magnetic resonance treatment; and
outputting the magnetic resonance treatment to a patient.

2. The method of claim 1, wherein the magnetic resonance treatment is output in the patient's home.

3. The method of claim 1, wherein the magnetic resonance application comprises a network connection to a remote database of magnetic resonance treatment protocols.

4. The method of claim 3, wherein the magnetic resonance treatment protocol is stored in the remote database of magnetic resonance treatment protocols.

5. The method of claim 1, wherein the magnetic resonance treatment protocol is developed by a magnetic resonance provider based on one or more prior magnetic resonance treatments to the patient.

6. The method of claim 1, wherein the magnetic resonance treatment protocol is developed by a magnetic resonance provider based in part on a database of data associated with other patients that have received magnetic resonance treatments.

7. The method of claim 1, wherein the magnetic resonance treatment protocol is developed by the patient.

8. The method of claim 1, wherein the magnetic resonance treatment comprises a magnetic field strength within the range of 1 gauss to 10⁻⁵⁰ gauss.

9. A magnetic resonance home unit comprising:
a magnetic resonance driver configured to receive a magnetic resonance treatment protocol and output a magnetic resonance treatment to a patient based in part on the magnetic resonance treatment protocol;
a first coil assembly configured to be driven by the magnetic resonance driver; and
a second coil assembly coupled to the first coil assembly, the second coil assembly configured to be driven by the magnetic resonance driver, the first and second coil assemblies configured to be collapsible.

10. The system of claim 9, wherein the magnetic resonance home unit is configured to be used by the patient in the patient's home.

11. The system of claim 9, wherein the magnetic resonance driver is configured to receive the magnetic resonance treatment protocol from a magnetic resonance application.

12. The system of claim 11, wherein the magnetic resonance application comprises a network connection to a remote database of data associated with magnetic resonance treatment protocols.

13. The system of claim 12, wherein the magnetic resonance treatment protocol is stored in the remote database.

14. The system of claim 9, wherein the magnetic resonance treatment protocol is developed by a magnetic resonance provider based on one or more prior magnetic resonance treatments to the patient.

15. The system of claim 9, wherein the magnetic resonance treatment protocol is developed by a magnetic resonance provider based in part on a database of data associated with other patients that have received magnetic resonance treatments.

16. The system of claim 9, wherein the magnetic resonance treatment protocol is developed by the patient.

17. The system of claim 9, wherein the magnetic resonance treatment comprises a magnetic field strength within the range of 1 gauss to 10⁻⁵⁰ gauss.

18. A non-transient computer readable medium comprising program code, which when executed by a processor, is configured to cause the processor to:
open a magnetic resonance application;
access a magnetic resonance treatment protocol associated with a magnetic resonance treatment; and
output the magnetic resonance treatment to a patient.

19. The non-transient computer readable medium 18, further comprising program code, which when executed by a processor, is configured to cause the processor to open a magnetic resonance application configured to access a network connection to a remote database of data associated with magnetic resonance treatment protocols.

20. The non-transient computer readable medium 18, wherein the magnetic resonance treatment comprises a magnetic field strength within the range of 1 gauss to 10⁻⁵⁰ gauss.
